(19) United States
(12) Patent Application Publication
(10) Pub. No.: US 2006/0046293 A1
(43) Pub. Date: Mar. 2, 2006

(54) Dopaminergic Nurr1-positive neuron stem cells, pharmaceutical composition therefor, and methods for isolation, culture and preservation thereof

(75) Inventor: Kuo-Liang Yang, Hualien (TW)

Correspondence Address: EDWARDS & ANGELL, LLP, P.O. BOX 55874, BOSTON, MA, 02205, US

(73) Assignee: Buddhist Tzu Chi General Hospital, Hualien (TW)

(21) Appl. No.: 10/974,273

(22) Filed: Oct. 26, 2004

(30) Foreign Application Priority Data
Sep. 1, 2004 (TW) 093126321

(51) Int. Cl. C12N 5/08; A61K 35/30

(52) U.S. Cl. 435/368; 424/93.7

(57) ABSTRACT

The present invention relates to a population of Nurr1-positive neuron stem cells, a pharmaceutical composition thereof, and methods for isolation, culture and preservation thereof. More specifically, the present invention provides an isolation method of Nurr1-positive neuron stem cells from human teeth. The said Nurr1-positive neuron stem cells can be applied in the treatment and/or prophylaxis of the Nurr1-related neurodegenerative diseases such as Parkinson's disease and stroke. The present invention further provides culturing and preservation methods of the said Nurr1-positive neuron stem cells.

### FIELD OF THE INVENTION

The invention relates to Nurr1-positive neuron stem cells, pharmaceutical compositions thereof, and methods for isolation, culture and preservation thereof, and more particularly, to isolation, culture and preservation of Nurr1-positive neuron stem cells from human teeth.

### BACKGROUND OF THE INVENTION

There is no promised drug available for the treatment of progressive disorder of the central nervous system or neurodegenerative diseases such as Parkinson's disease, Huntington's disease and Alzheimer's disease. For Parkinson's disease, the ideal treatment is to provide a neural protection therapy to a patient after being diagnosed having the Parkinson's disease. In general, the standard healthcare for the Parkinson's patient is to administer levodopa or dopamine agonist to the patient so as to alleviate or inhibit disease progress. Levodopa is converted to dopamine that is supplemented to an insufficient amount of dopamine in the patient's brain. However, levodopa cannot prevent the progressive pathological changes of the brain of the Parkinson's patient. Further, levodopa may be converted to dopamine before reaching the brain, such that long-term administration of levodopa would cause a motor complication such as dyskinesia or motor fluctuation. It has been proven that dopamine agonist can alleviate symptoms of the Parkinson's disease. To avoid the motor complication, dopamine agonist may be used for the therapy in the early stage of the Parkinson's disease. However, along with the progression of such disease, single medication with dopamine agonist is practically not sufficient to alleviate the disease symptoms, and cooperation of dopamine agonist and levodopa is required to effectively control the symptoms.

It has been proposed to use stem cells for developing cell-based therapy. The stem cells include totipotent stem cells and pluripotent stem cells. Totipotent stem cells are referred to the cells each capable of growing to a complete organism, such as blastocyte cells. Pluripotent stem cells have the limited ability and differentiate to specific tissue stem cells depending on functions thereof, such as hematopoietic stem cells, neural stem cells, skin stem cells and the like. Currently, most of the stem cells used in therapy are collected from bone marrow and cord blood, and as restricted by functions thereof, are primarily applied to hematological malignant diseases, inborn metabolic diseases and immune deficient diseases and to recovery of bone marrow functions that have been damaged by chemotherapy or radiotherapy. Roy et al. have successfully isolated stem cells from the human brain (hippocampus) due to the fact of neurodegeneration of neuron cells in the brain hippocampus of Alzheimer's patients (Nature Medicine 2000; 6:249-250,271-277). However, this paper does not disclose whether or not the isolated stem cells are Nurr1-positive stem cells.

It has been proposed to isolate stem cells from pulp cells. Gronthos et al. have provided an isolation method of stem cells from impacted third molar (Proceedings of the National Academy of Science of the United State of America; PNAS, 2000; 97(25): 13625-13630). This method comprises preparing pulp tissues from cementum-enamel junctions, treating the tissues with collagenase and dispase, and obtaining stem cells by filtration. Miura et al. have collected and cultured pulp from normal exfoliated human deciduous teeth of 7- or 8-year-old children, and identified stems cells by using specific cell markers of stem cells (PNAS 2003; 100(10): 5807-5812). They have found that about 12 to 20 stem cells, customarily named SHED (stem cells from human exfoliated deciduous teeth), can be obtained from an incisor. Kuo et al. have isolated stem cells from dental papilla, and the isolated stem cells are converted to dopamine-producing cells through trans-differentiation by transfection of Nurr1 gene to such cells (JBSC; April, 2004).

For Parkinson's disease, it may be induced when the neuron cells in substantia nigra area of the brain are damaged. These brain neuron cells are responsible for producing a neurotransmitter, dopamine, which allows neural messages to control muscle activities. The production of brain dopamine is related to a group of transcription factors/nuclear receptors named Nurr1. The mechanism is to induce Nurr1 expression by membrane depolarization to increase the concentration of Nurr1 mRNA in PC12 brain cells. Ramsden et al. have found that the aetiology of idiopathic Parkinson's disease is related to dopaminergic neurogenesis factors such as Nurr1, Ptx-3 and Lmx1b (Mol. Pathol. 2001 December; 54(6): 369-380). U.S. Pat. No. 2003/0119026A1 to Le and Vassilatis discloses a diagnostic method for Parkinson's disease, which uses mutation of Nurr1 polypeptide (i.e. mutation on Nurr1 gene). In other words, abnormal change (mutation) on Nurr1 gene results in the disorder of dopamine concentration in the brain cells, thereby inducing the neurodegenerative diseases such as Parkinson's disease. Therefore, it is desired to easily and sufficiently obtain and culture neuron stem cells with Nurr1 cell marker to be used in therapy or drug development for such neurodegenerative diseases.

However, since Nurr1-positive neuron stem cells are only known existing in mesencephalon, it is difficult to obtain tissue culturing material of these neuron stem cells in vivo. Therefore, the problem to be solved here is to develop a simple, fast and efficient method for isolation, culturing and preservation of Nurr1-positive neuron stem cells, which facilitates the therapy and development of drugs for Nurr1-associated neurodegenerative diseases.

### SUMMARY OF THE INVENTION

To solve the foregoing problems in the prior art, the present invention provides a simple, fast and efficient isolation method of neuron stem cells. More specifically, the present invention provides a method of isolating Nurr1-positive neuron stem cells from human impacted molar teeth. This method to obtain neuron stem cells comprises the steps of: 1) rinsing a prepared sample of molar teeth with normal saline; 2) placing the rinsed molar teeth sample in a 50 mL conical test tube containing 30 mL of clean normal saline, and vigorously shaking it to produce a suspension of cells; 3) subjecting the cell suspension obtained in the step (2) to centrifugation; 4) after centrifugation, decanting supernatant and suspending the pellet cells in Medium 199 (Gibco) containing 5 to 10% FBS (fetal bovine serum) to have a concentration of cells of 1×10⁶ to 3×10⁶/mL, such that the cell suspension is transferred to a 25 cm² tissue culture flask having 4 to 5 mL of Medium 199 (Gibco) containing 5 to 10% FBS; and 5) culturing the cells in the tissue culture flask at a 37° C. incubator supplied with 5% CO₂ for 4 to 10 days so as to obtain the neuron stem cells. Fresh Medium 199 containing 5 to 10% FBS is replaced at day 5 or 6 and every 3 to 4 days thereafter, which may or may not contain penicillin and streptomycin (Biowest). According to this method, the primary neuron stem cells being isolated from human molar teeth can be cultured for over 10 passages.

A primary neuron stem cell line (Nurr1-positive) was deposited with the BCRC (Bioresource Collection of Research Center, 331 Shih-Pin Road, Hsinchu, 300 Taiwan, R.O.C.) on Jul. 6, 2004, and assigned Accession No. BCRC 960209.

The present invention also provides a culturing method of frozen-stored neuron stem cells, comprising the steps of: 1) removing a vial of the frozen-stored cells from freezer and placing the vial in a 37° C. water bath to thaw the cells; and 2) transferring a suspension of the thawed cells to a 25 cm² tissue culture flask, and culturing the cells in 4 to 5 mL of Medium 199 (Gibco) containing 5 to 10% FBS at a 37° C. incubator supplied with 5% CO₂ for 4 to 5 days.

The present invention further provides a preservation method of neuron stem cells, comprising the steps of: 1) culturing the neuron stem cells to be 85% confluence in a tissue culture flask and stripping the cells from the tissue culture flask by means of an optimal volume of trypsin-EDTA (ethylenediaminetetraacetate); 2) collecting 2 mL of a suspension of the cells having a concentration of 1×10⁵ to 5×10⁵/mL into a 15 mL conical test tube, and adding 12 mL of normal saline to the cell suspension; 3) subjecting the cell suspension to centrifugation; 4) after centrifugation, decanting supernatant and repeating the step of adding normal saline to the cell suspension and the step (3); 5) after decanting the supernatant, adding 0.5 mL of Medium 199 containing 5 to 10% FBS to the pellet cells obtained by centrifugation and mixing them thoroughly to suspend the cells; 6) adding 0.5 mL of a solution of 55% w/v DMSO (dimethyl sulfoxide) with 5% dextran40 to the cell suspension obtained in the step (5), wherein the solution acts as a cryoprotectant; and 7) distributing a mixture of the cells and the cryoprotectant in 0.5 mL aliquot into freezing vials and storing the vials at −80° C. freezer until use.

### BRIEF DESCRIPTION OF THE DRAWINGS

The present invention can be more fully understood by reading the following detailed description of the preferred embodiments, with reference made to the accompanying drawings, wherein:

FIG. 1 is a light microscopic photograph of Nurr1-positive neuron stem cells isolated according to the invention;

FIG. 2 is another light microscopic photograph of the Nurr1-positive neuron stem cell isolated according to the invention;

FIG. 3 is a further light microscopic photograph of the Nurr1-positive neuron stem cell isolated according to the invention;

FIG. 4 is a photograph of the neuron stem cells isolated in the invention being subjected to immune fluorescence cytostaining in the use of primary antibodies against β₃-tublin, wherein the neuron stem cells have antigens binding to the antibody against β₃-tublin and show fluorescence in immunocytochemistry;

FIG. 5 is a photograph of the neuron stem cells isolated in the invention being subjected to immune fluorescence cytostaining in the use of primary antibodies against Nestin, wherein the neuron stem cells have antigens binding to the antibody against Nestin and show fluorescence in immunocytochemistry;

FIG. 6 is a photograph of RT-PCR electrophoresis showing the neuron stem cells isolated in the invention have mRNA of Nurr1 and is proved as Nurr1-positive neuron stem cells;

FIG. 7 is a photograph of RT-PCR electrophoresis showing the neuron stem cells isolated in the invention have mRNA of GAPDH, NFM and Nestin and do not have a cell marker (GFAP) of non-neuron stem cells; and

FIGS. 8(a) to 8(g) are photographs of the Nurr1-positive neuron stem cell culture isolated and cultured according to the invention, which are obtained by taking photographs once per day for 7 days on a cell culture from the same position of the same culture flask and show that the cells have a self-renewal characteristic.

FIGS. 9(a) to 9(c) depict HPLC-ECD (High Performance Liquid Chromatography-Electro Chemical Detection) analyses of the tissue culture media of Nurr1-positive neuron stem cells. Comparisons of HPLC-ECD (High Performance Liquid Chromatography-Electro Chemical Detection) of 0.05 ng/ul catecholamine standard solution (FIG. 9a), tissue culture media of Nurr1-positive neuron stem cells (FIG. 9b) and fresh un-used medium (FIG. 9c) indicate that the Nurr1-positive neuron stem cells have a property to release dopamine in the tissue culture conditions described herewith.

### DETAILED DESCRIPTION OF THE PREFERRED EMBODIMENTS

Isolation and Culturing of Neuron Stem Cells

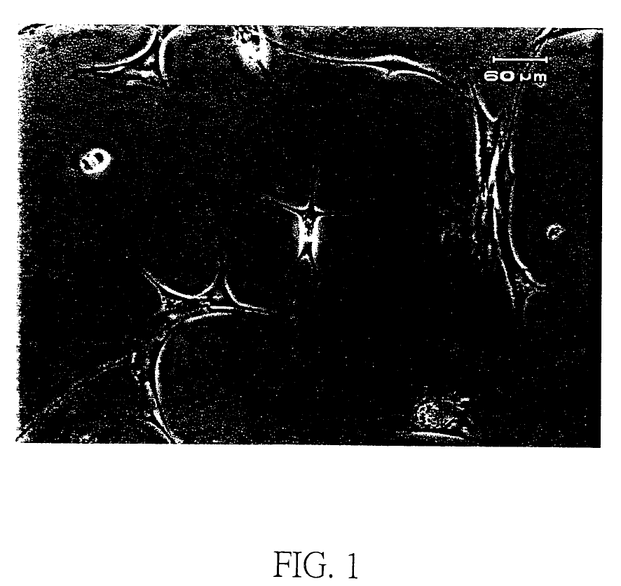

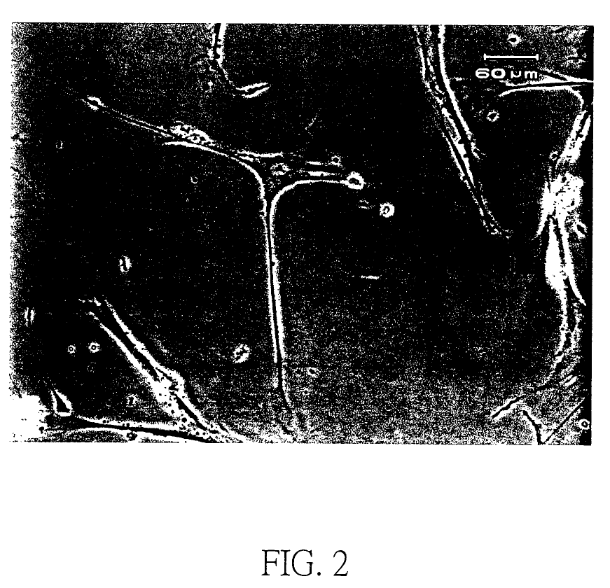

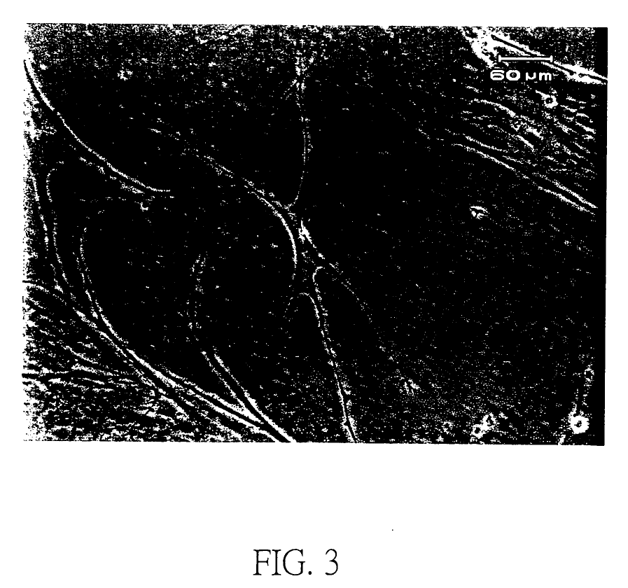

With full consents from three donors, each of an age between 19 and 25 years old, according to approved study guidelines from the Department of Health in Taiwan, impacted molar teeth are collected from the three donors and rinsed with normal saline. The rinsed molar teeth sample is put into in a 50 mL conical test tube containing 30 mL of normal saline, and vigorously shaken or vortexed for 30 minutes to produce a suspension of cells. The cell suspension is subjected to centrifugation at 2000 rpm for 10 minutes. After decanting supernatant, the pellet cells are suspended in Medium 199 (Gibco, a tissue culture medium) containing 5 to 10% FBS (fetal bovine serum) to have a concentration of cells of 1×10⁶ to 3×10⁶/mL, and the cell suspension is transferred to a 25 cm² tissue culture flask having 4 to 5 mL of Medium 199 containing 5 to 10% FBS. The cells are cultured at a 37° C. incubator supplied with 5% CO₂ atmosphere for 4 to 10 days and began to appear with morphology of neuron stem cells. As shown in FIGS. 1 to 3, the isolated cells have the morphology of neuron stem cells. Fresh Medium 199 containing 5 to 10% FBS is replaced at day 5 or 6 and every 3 to 4 days thereafter, which may or may not contain penicillin and streptomycin (Biowest). According to this method, the primary neuron stem cells being isolated from human molar teeth can be cultured for over 10 passages.

Freezing and Thawing of Neuron Stem Cells

The neuron stem cells are cultured to be 85% confluence in a tissue culture flask and stripped from the tissue culture flask by means of an optimal volume of trypsin-EDTA (ethylenediaminetetraacetate). To facilitate the removal of the adherent cells, cells treated with trypsin may be incubated in a 37° C. incubator for 10 to 15 minutes. After incubation, 2 mL of the cell suspension with a concentration of 1×10⁵ to 5×10⁵/mL is collected into a 15 mL conical test tube and added with 12 mL of normal saline. Then, the cell suspension is subjected to centrifugation at 1800 rpm for 10 minutes. After centrifugation, supernatant is decanted, and the above steps of adding normal saline and centrifugation are repeated. After decanting the supernatant, 0.5 mL of Medium 199 containing 5 to 10% FBS is added to the pellet cells obtained by centrifugation and mixed with the cells thoroughly to form a cell suspension. 0.5 mL of a solution of 55% w/v dimethyl sulfoxide (DMSO) with 5% dextran40 is added to the cell suspension, wherein this solution acts as a cryoprotectant. A mixture of the cells and the cryoprotectant is then distributed in 0.5 mL aliquot into freezing vials that are stored immediately at −80° C. freezer until use.

To thaw the frozen-stored cells, a vial of the cells is removed from the freezer and placed in a 37° C. water bath to quickly thaw the cells. The thawed cell suspension is then transferred to a 25 cm² tissue culture flask having 4 to 5 mL of Medium 199 containing 5 to 10% FBS and is incubated at a 37° C. incubator supplied with 5% CO₂.

Immunocytochemistry

For identification of cell type specific antigens, an immune fluorescence cytostaining process is performed. The neuron stem cells isolated in the present invention are seeded overnight in 24-well culture plate with 1×10⁴ cells per well and fixed with a pre-chilled solution of acetone:methanol (1:1) for 15 min at −20° C. Nonspecific binding is blocked with normal serum from the species which is used for the secondary antibodies production (10% secondary antibodies in PBS containing 0.25% Triton X-100). The fixed neuron stem cells are incubated with primary antibodies diluted in PBS containing 0.25% Triton X-100 for 2 hours at 25° C. After being rinsed with PBS, the neuron stem cells are incubated with FITC- or rhodamine-conjugated secondary antibodies for 1 hour at the room temperature in dark and then observed with Axiovert 200M (Zeiss) fluorescence-inverted microscope. The observed images are taken and processed by Metamorph (Universal Imaging Co. Ver 6.0 rev 5) equipped with CoolSnap HQ CCD camera. In the control wells, all the above procedures are performed except the use of the primary antibodies. The primary antibodies used in this study include: anti-CD34 (1:200; Pharmingen), anti-CD45 (1:200; Pharmingen), anti-CD90 (1:200; Pharmingen), anti-GFAP (1:200; Sigma), anti-Nestin (1:400; Chemicon), anti-β₃-tublin, and anti-human β2 microglobulin (1:400; Santa Cruz). Cell types for the above primary antibodies are listed in Table 1.

TABLE 1

| Primary Antibody | Cell Type |
|---|---|
| anti-CD34 | Hematopoietic stem cell |
| anti-CD45 | White blood cell |
| anti-CD90 | Leukocyte |
| anti-GFAP | Glial cell |
| anti-Nestin | Neural progenitor |
| anti-β₃-tublin | Neuron |
| anti-human β₂ microglobulin | β₂ microglobulin |

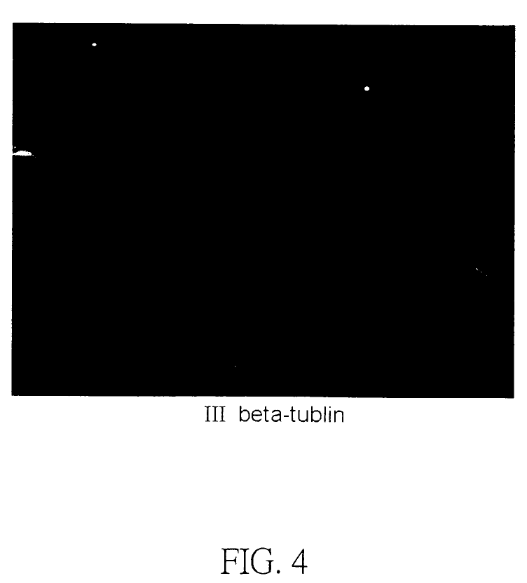

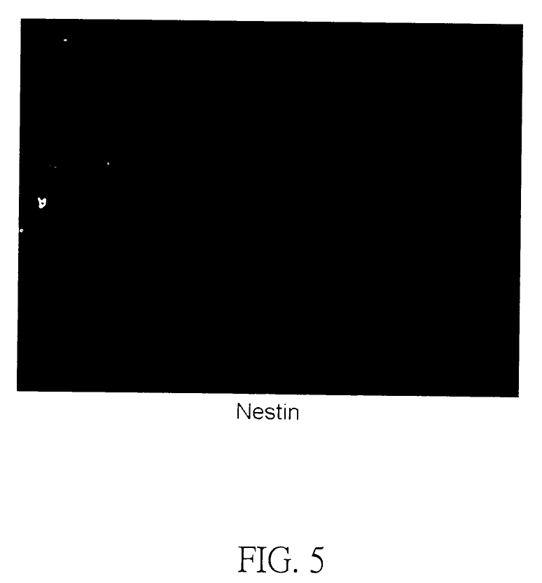

The results are shown in FIGS. 4 and 5 that the neuron stem cells isolated in the present invention do have cell markers (Nestin and β₃-tublin) of neuron stem cells but no cell marker (GFAP) of non-neuron stem cells.

Reverse Transcription-Polymerase Chain Reaction (RT-PCR)

RNA (ribonucleic acid) is extracted using RNeasy kit (Qiagen, GmBH) from the neuron stem cells isolated in this invention that are grown to 80% confluence in a 75T tissue culture flask. Messenger RNA (mRNA) is reverse-transcribed using Omniscript RT (Qiagen GmBH) to complementary DNA (cDNA), and one tenth of the cDNA is subjected to 30 cycles of PCR amplification (ABI PRISM 9700, Applied Biosystems) with the following conditions/parameters for PCR.
1. Initial denaturation at 95° C. for 10 minutes
2. Denaturation at 95° C. for 30 seconds
3. Annealing at 55° C. for 30 seconds
4. Elongation at 72° C. for 60 seconds
5. Repeat Steps 2 to 4 for 30 cycles

Controls are performed with the amplification reaction without addition of cDNA template and without reverse transcription. The authenticity and sizes of PCR products are confirmed by electrophoresis analysis. The sizes of expected PCR products corresponding to primers used are shown in Table 2.

TABLE 2

| Cell Marker | Size of Expected Products | Cell Type |
|---|---|---|
| Nurr1 | 712 bp | Neuron stem cell |
| GAPDH | 251 bp | Housekeeping gene |
| NFM | 204 bp | Neuron stem cell |
| Nestin | 197 bp | Neural progenitor |
| GFAP | 226 bp | Glial cell |
| Tau | 204 bp | Axon protein |

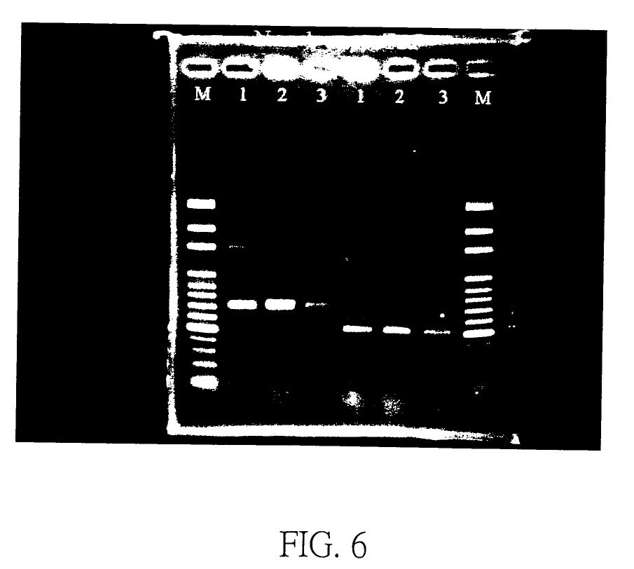

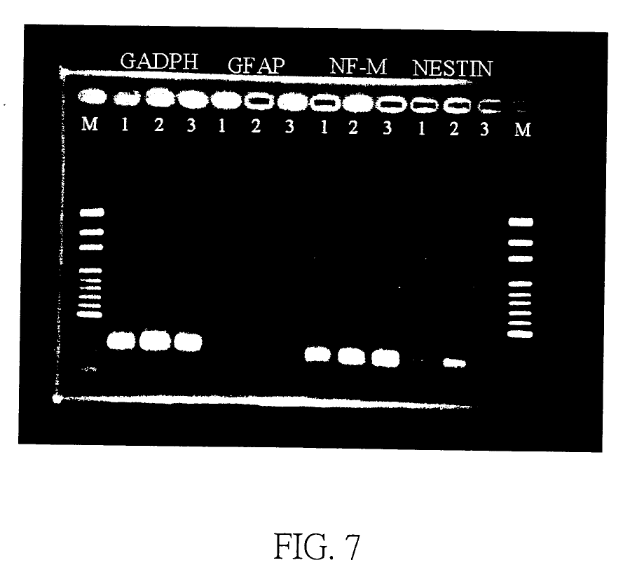

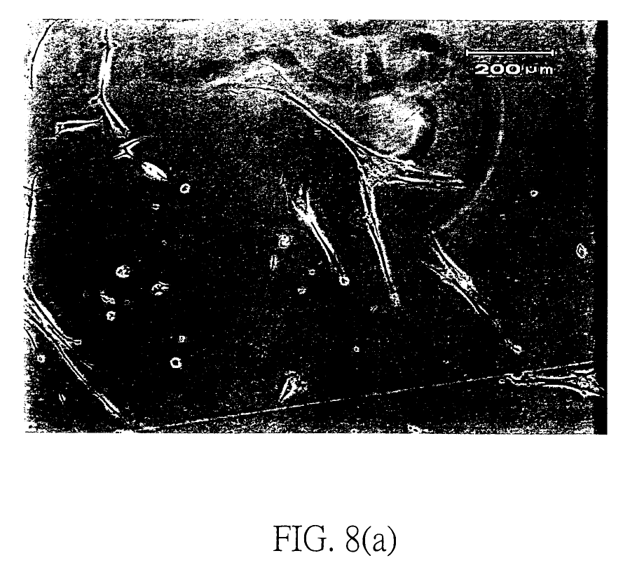

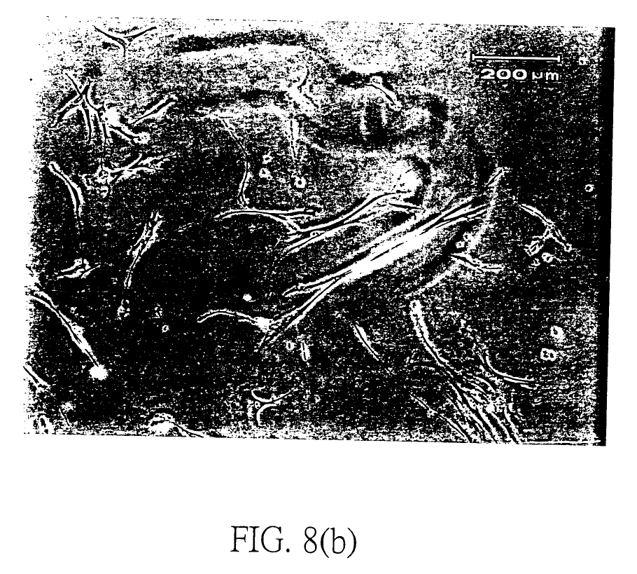

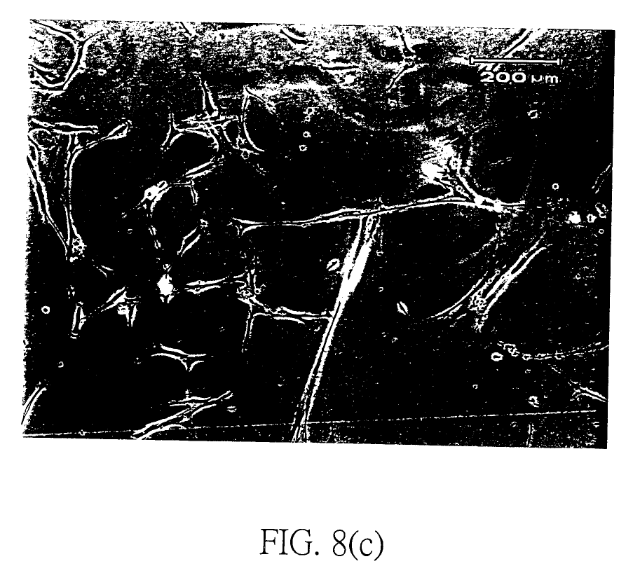

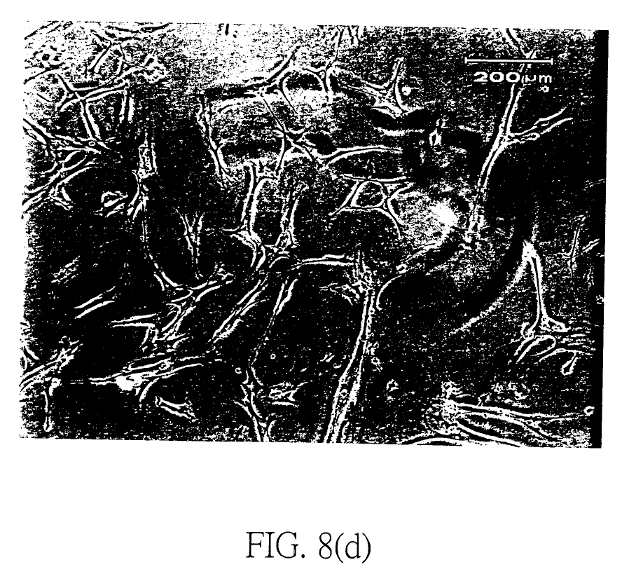

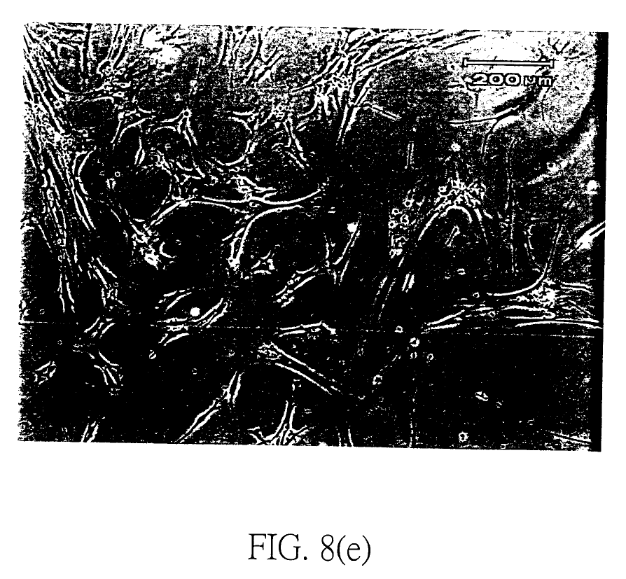

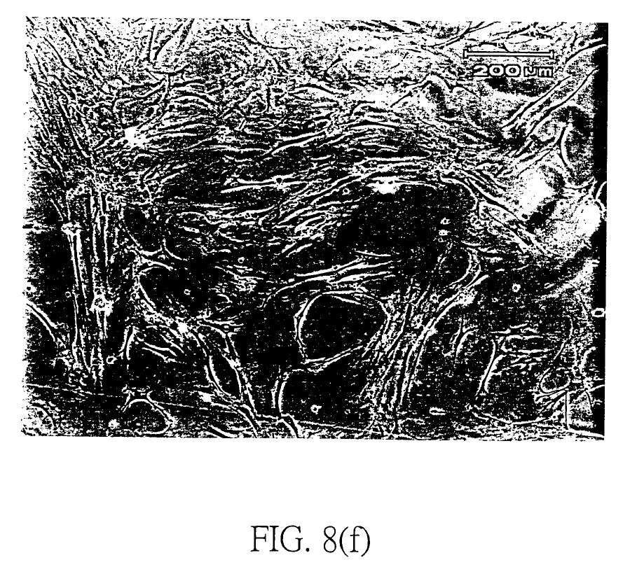

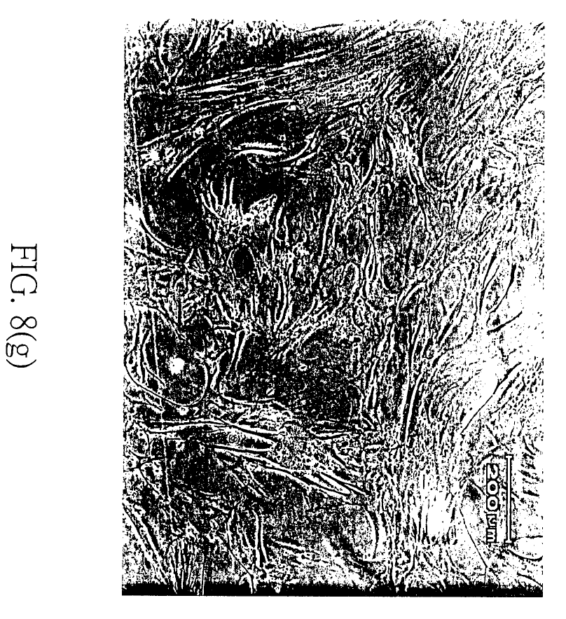

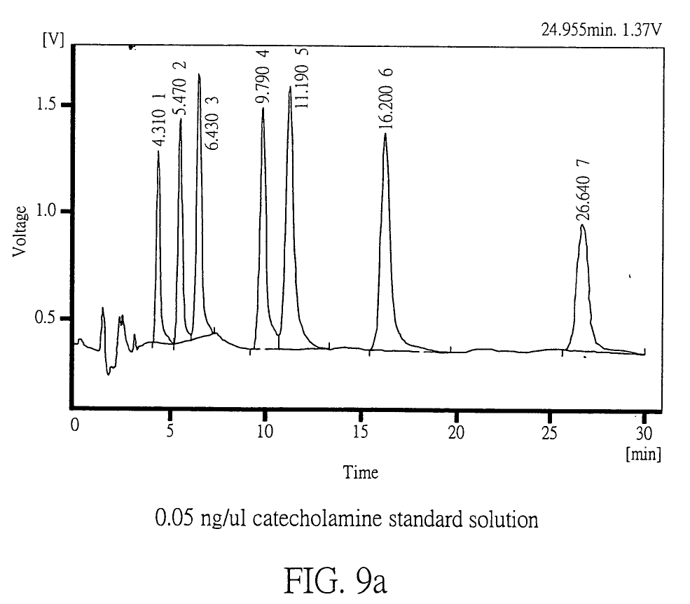

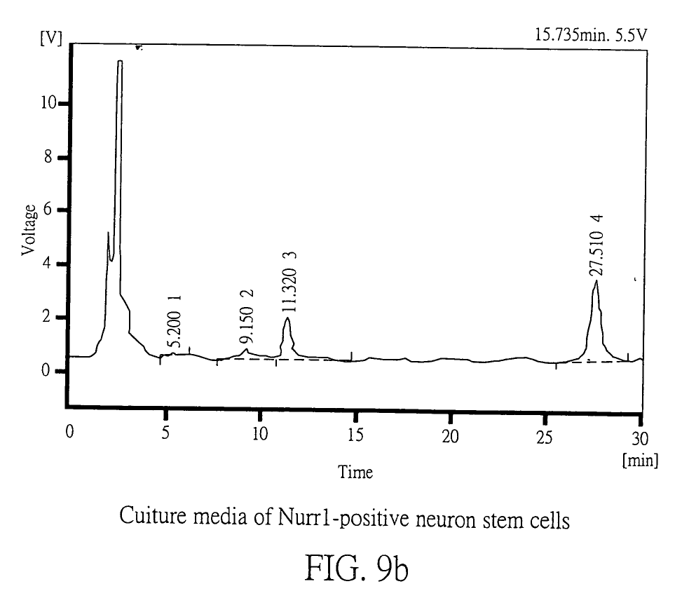

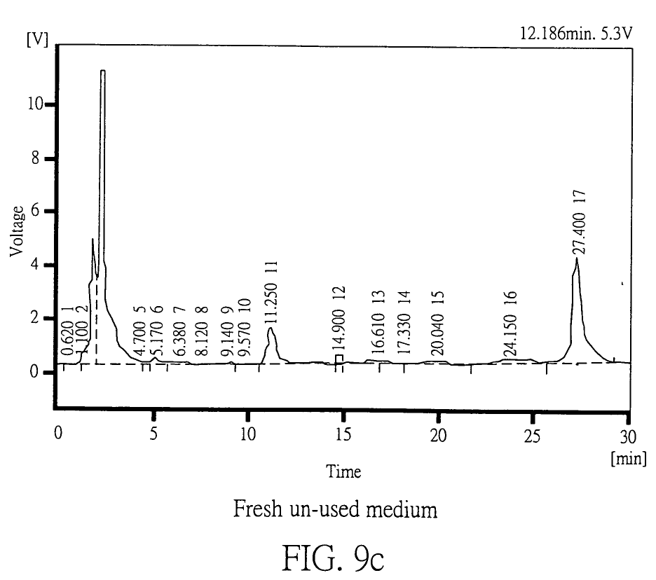

Before amplification, the mRNA levels of the samples are normalized by GAPDH as housekeeping gene. FIGS. 6 and 7 show the results of electrophoresis analysis that the neuron stem cells isolated in the present invention do have cell markers (Nurr1, NFM and Nestin) of neuron stem cells but not a cell marker (GFAP) of non-neuron stem cells.

From the above results, the isolation method according to the present invention, the cells isolated from different donors' molar teeth are proved as neuron stem cells, which can be Nurr1-positive cells. The present invention not only provides a simple and fast isolation method of neuron stem cells but also allows Nurr1-positive neuron stem cells to be isolated. It is known that Nurr1 neuron stem cells primarily reside in the brain. However, for the drug development, etiology study, treatment and cell-based therapy for Nurr1-related neurodegenerative diseases, a simple and fast method to obtain Nurr1-positive cell lines is deemed necessary. Therefore, according to the method in the present invention, primary Nurr1-positive neuron stem cells can be easily obtained without using complicated surgical operations, and the obtained primary Nurr1-positive neuron stem cells can be cultured for subsequent passages and preserved by simple methods.

The invention has been described using exemplary preferred embodiments. However, it is to be understood that the scope of the invention is not limited to the disclosed embodiments. On the contrary, it is intended to cover various modifications and similar arrangements. The scope of the claims, therefore, should be accorded the broadest interpretation so as to encompass all such modifications and similar arrangements.

What is claimed is:

1. A population of Nurr1-positive neuron stem cells, which is obtained by isolation from human teeth.

2. The population of Nurr1-positive neuron stem cells of claim 1, wherein the human teeth are molar teeth.

3. An isolation method of Nurr1-positive neuron stem cells, comprising:
(1) preparing a sample of human teeth and rinsing the human teeth sample with normal saline;
(2) placing the rinsed human teeth sample in clean normal saline, and shaking it to produce a suspension of cells;
(3) subjecting the cell suspension obtained in the step (2) to centrifugation;
(4) after centrifugation, decanting supernatant, and suspending the pellet cells in a proper medium to have a predetermined concentration of cells, such that the cell suspension is transferred to a tissue culture flask; and
(5) culturing the cells in the tissue culture flask at an incubator for 4 to 10 days so as to obtain the Nurr1-positive neuron stem cells.

4. The isolation method of claim 3, wherein the human teeth are molar teeth.

5. A pharmaceutical composition for treatment of a neurodegenerative disease, comprising the Nurr1-positive neuron stem cells of claim 1 as the effective ingredient.

6. The pharmaceutical composition of claim 5, wherein the neurodegenerative disease is Parkinson's disease or stroke.

7. A culturing method of frozen-stored Nurr1-positive neuron stem cells, comprising:
(1) removing a vial of the frozen-stored Nurr1-positive neuron stem cells from freezer and placing the vial in a 37° C. water bath to thaw the cells; and
(2) transferring a suspension of the thawed cells to a tissue culture flask, and culturing the cells in a medium containing 5 to 10% FBS (fetal bovine serum) at an incubator.

8. The culturing method of claim 7, wherein the Nurr1-positive neuron stem cells are obtained by isolation from human teeth.

9. The culturing method of claim 8, wherein the human teeth are molar teeth.

10. A freezing preservation method of Nurr1-positive neuron stem cells, comprising:
(1) culturing the neuron stem cells to be 85% confluence in a tissue culture flask and stripping the cells from the tissue culture flask by means of an optimal volume of trypsin-EDTA (ethylenediaminetetraacetate);
(2) collecting a suspension of the cells and adding normal saline to the cell suspension;
(3) subjecting the cell suspension to centrifugation;
(4) after centrifugation, decanting supernatant, and repeating the steps (2) and (3) for at least one time;
(5) after decanting the supernatant, adding a proper medium to the pellet cells obtained by centrifugation and mixing them thoroughly to suspend the cells;
(6) adding a cryoprotectant of DMSO(dimethyl sulfoxide)/dextran to the cell suspension obtained in the step (5); and
(7) distributing a mixture of the cells and the cryoprotectant into freezing vials and storing the vials at −80° C. freezer until use.

11. The freezing preservation method of claim 10, wherein the cells are treated with trypsin-EDTA at 37° C. for 10 to 15 minutes.

12. The freezing preservation method of claim 10, wherein the neuron stem cells are obtained by isolation from human teeth.

13. The freezing preservation method of claim 12, wherein the human teeth are molar teeth.
